(19) United States
(12) Patent Application Publication
(10) Pub. No.: US 2008/0228088 A1
(43) Pub. Date: Sep. 18, 2008

(54) System and Method for Detecting and Predicting a Syncope Event

(75) Inventors: Xavier Aubert, Brussels (BE); Jens Muhlsteff, Aachen (DE); Michael Perkuhn, Aachen (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 12/064,998

(22) PCT Filed: Aug. 23, 2006
(86) PCT No.: PCT/IB2006/052917

§ 371 Date: Feb. 27, 2008

(30) Foreign Application Priority Data
Aug. 31, 2005 (EP) 05107948.1

(51) Int. Cl. A61B 5/02

(52) U.S. Cl. 600/485; 600/504

(57) ABSTRACT

The present invention relates to a system (1) and method for detecting and predicting a syncope event. Furthermore the invention relates to a computer program (13) to be executed in a computer (5, 17), said computer (5, 17) being part of a system (1) for detecting and predicting a syncope event. It is an object of the present invention to provide a simple and reliable technique for detecting and predicting the occurrence of syncope events. This object is achieved according to the invention by a method of detecting and predicting a syncope event, the method comprising the steps of continuously obtaining (100) the pulse wave patterns (3) from the upper vascular system of the user (4), determining (101) from the pulse wave patterns (3) a measure (21) of the blood flow to the user's brain, and generating (103) an alarm signal (6), if the blood flow measure indicates the future occurrence of a syncope.

The present invention relates to a system and method for detecting and predicting a syncope event. Furthermore the invention relates to a computer program to be executed in a computer, said computer being part of a system for detecting and predicting a syncope event.

A syncope is a sudden, transient and reversible loss of consciousness. It is an important medical problem because it is common, costly, often disabling (hospitalization in 50% of the cases) and may cause severe injuries, especially among elderly people. In the US, syncope events represent around 5% of the costs in emergency units and around 6% of all hospital admissions. The overall costs of evaluating and treating patients with syncope has been estimated to US-$ 800 million per year in 1999.

Syncope events arise from the inability to maintain a central volume of circulating blood, leading to an insufficiency of cerebro-vascular blood perfusion. There is no unique explanation of syncope, rather, a number of possible causes originating from the cardiac, vascular or neurological levels. The most frequent identified causes are either cardiac arrhythmias (abnormal heart-rate variations, bradycardia) or inadequate neural reflexes inducing a vaso-vagal syncope. The latter covers 50 to 90% of the syncopes by young patients and is due to inappropriate baro-receptor feedbacks leading to an insufficient return of venous blood from the lower limbs in upright position, such that the blood pressure cannot be maintained high enough in the brain.

In spite of this complex and diverse etiology, the majority of syncope events follows from an insufficient blood supply to the brain. Existing devices dealing with syncope detection or prevention are mostly embedded in (implantable) pacemakers taking care of heart rate regulation. Hence, syncope events caused by a pressure drop not related to cardiac arrhythmias cannot be handled.

It is an object of the present invention to provide a simple and reliable technique for detecting and predicting the occurrence of a syncope event.

This object is achieved according to the invention by a method comprising the steps of continuously obtaining the pulse wave patterns from the upper vascular system of the user, determining from the pulse wave patterns a measure of the blood flow to the user's brain, and generating an alarm signal, if the blood flow measure indicates the future occurrence of a syncope.

The object of the present invention is also achieved by a system for detecting and predicting a syncope event, the system comprising a sensor unit adapted for continuously obtaining the pulse wave patterns from the upper vascular system of the user, a processing unit adapted for determining from the pulse wave patterns a measure of the blood flow to the user's brain, said processing unit being further adapted for generating an alarm signal, if the blood flow measure indicates the future occurrence of a syncope.

The object of the present invention is also achieved by a computer program to be executed in a computer, said computer being part of a system for detecting and predicting a syncope event, said system comprising a sensor unit adapted for continuously obtaining the pulse wave patterns from the upper vascular system of the user, said program comprising computer instructions to determining from the pulse wave patterns a measure of the blood flow to the user's brain, when the computer program is executed in the computer. The technical effects necessary according to the invention can thus be realized on the basis of the instructions of the computer program in accordance with the invention. Such a computer program can be stored on a carrier such as a CD-ROM or it can be available over the internet or another computer network. Prior to executing the computer program is loaded into the computer by reading the computer program from the carrier, for example by means of a CD-ROM player, or from the internet, and storing it in the memory of the computer. The computer includes inter alia a central processor unit (CPU), a bus system, memory means, e.g. RAM or ROM etc., storage means, e.g. floppy disk or hard disk units etc. and input/output units. Alternatively, the inventive method could be implemented in hardware, e. g. using one or more integrated circuits.

A core idea of the invention is to provide a technique for closely and permanently observing the blood perfusion to the brain of a user for ambulatory use. At the same time the system employed is adapted to exhibit a very high wearing comfort. Since the occurrence of syncope is predicted and the user is warned before an insufficient blood supply leads to unconsciousness, appropriate actions can be taken like sitting or lying down to prevent from falling on the ground, which often is combined with severe consequences, e.g. fall injuries.

The upper vascular system comprises the vascular system of the upper part of the user's body. The upper vascular system comprises e.g. a carotid artery, preferably in the upper neck region of the user, and a subclavian artery in the shoulder region of the user.

These and other aspects of the invention will be further elaborated on the basis of the following embodiments which are defined in the dependent claims.

According to a preferred embodiment of the invention the pulse signal of one of the carotid or subclavian arteries is continuously monitored in order to obtain the pulse wave patterns. In other words there is no direct measuring of the blood flow. This allows the use of small, light and unobtrusive sensors.

For monitoring the pulse signal of the upper vascular system the sensor unit preferably comprises a passive sensor, in particular a piezo-foil transducer adapted for measuring the surface vibrations of the user's upper vascular system arterial pulse.

Since no active sensor is used, i.e. the sensor does not emit a radio frequency signal or the like, there is no negative effect to the user's health. In particular there is no heating effect on the user's tissues surrounding the sensor.

Furthermore this kind of sensor need less energy as other sensor types. That is the sensor does not require a power supply that could not be provided by normal batteries for ambulatory use over a long time period, e.g. over a period of several hours. This makes the sensor well suited for long-term ambulatory monitoring.

The power supply (e.g. a battery) for the sensor is advantageously provided within the sensor unit. Thus a disturbing cabling is not needed.

For the purpose of the present invention preferably a single sensor is used for monitoring the carotid pulse signal. Such a single sensor arrangement leads to an enhanced wearing comfort. Furthermore a light sensor is used, e.g. having a weight of less than 10 g. The use of such a light sensor again improves the wearing comfort considerably.

In a preferred embodiment of the invention the measured data is transferred from the sensor unit to the processing unit by means of a wireless communication line. Since in this case movements of the user's head do not lead to tensions between the sensor and the transmitting wire, the data recording is not affected. Thus the absence of a wire connection between the two units reduces the motion artifacts. At the same time the wireless connection enhances the wearing comfort of the sensor unit.

According to a preferred embodiment of the invention the sensor is located on the carotid artery, preferably in the upper neck region of the user or on the subclavian artery in the shoulder region of the user. By this means it is ensured, that the way to the user's brain is short. Thus, possible deficiencies of the blood supply to the user's brain can be determined in a safe way. This approach requires an accurate positioning of the sensor on the artery. On the other hand, with this accurate positioning the use of a larger number of sensors can be avoided.

According to another preferred embodiment of the invention the determining of the measure of the blood flow is carried out by means of the processing unit based both on the heart rate and the stroke volume.

In other words, the employed sensor does not provide a direct measure of the blood flow. Instead, the blood flow is determined in an indirect way by continuously computing a measure from the pulse wave patterns of the user by means of the processing unit, said pulse wave patterns being continuously captured by means of the sensor unit. For the purpose of computing a measure of the blood flow, the deduced heart rate (pulse rate) and the pulse amplitude and/or pulse shape (i.e. blood pressure variations) as a measure of the stroke volume are algorithmically combined by means of the processing unit.

In other words, the pulse wave is analyzed by means of the processing unit and a single characteristic value is computed. Said characteristic value serves as a measure of the blood flow to the user's brain.

According to another preferred embodiment of the invention the alarm signal is generated by means of the processing unit, if the blood flow falls below a given critical level. The critical level is preferably set once the system is prepared for use. Alternatively the critical level is adapted automatically by means of the processing unit, taking into account previous measuring data of the user.

If the determined measure falls below the critical level, preferably an alarm sound is emitted to the user and/or an alarm message is transmitted to a remote receiving unit, e.g. to a medical emergency service.

The present invention suggests a technique for monitoring the blood flow variations to the brain, irrespective of the underlying physiological causes. Hence, the main types of syncope are dealt with, namely, those due to cardiac arrhythmias affecting the heart rate as well as those affecting the arterial blood pressure like the vaso-vagal syncope's.

The new technique according to the invention is not primarily meant for a quick check of the carotid pulse (although it could fulfill this goal), but for a continuous, long-term monitoring of the blood perfusion to the user's brain. Since a sudden drop of blood flow to the user's brain can be alerted in time, arrangements can be made accordingly. In other words the invention can be used as a prophylactic measure. Another feature offered by the invention is the possibility of recording the upper vascular system pulse signal such that post-syncope analyses can be undertaken by examining the recorded cardio-vascular signals and an enhanced diagnostic can be achieved. In other words the invention can also be used in a therapeutic approach.

These and other aspects of the invention will be described in detail hereinafter, by way of example, with reference to the following embodiments and the accompanying drawings; in which:

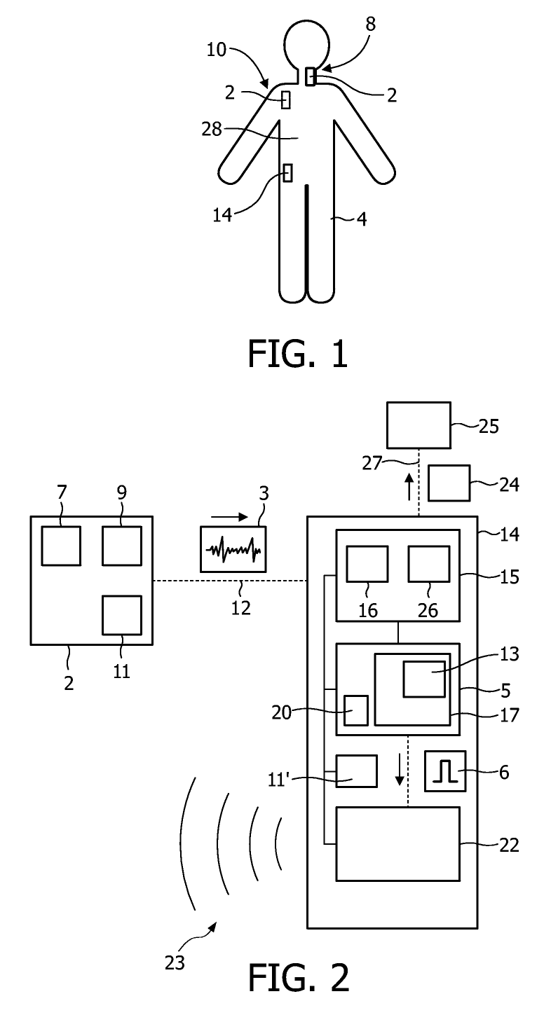

FIG. 1 shows a schematic illustration of a user wearing the sensor unit,

FIG. 2 shows a schematic block diagram of a system according to the invention,

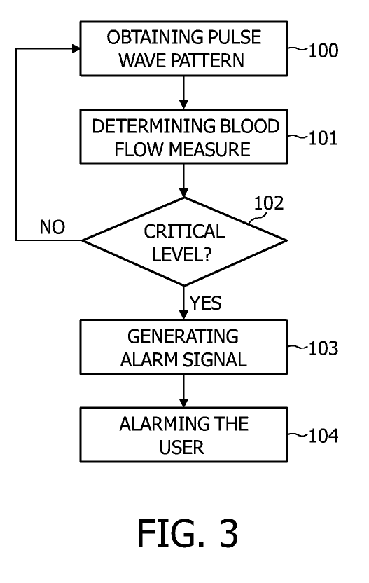

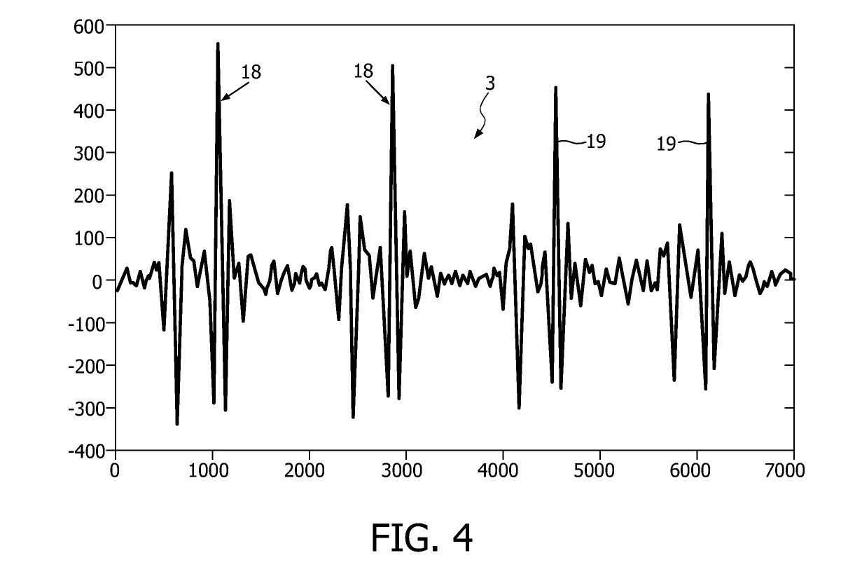

FIG. 3 shows a simplified flowchart of the method according to the invention, and

FIG. 4 shows a diagram with a piezo-foil transducer signal from left carotid artery of a male user.

Referring to FIGS. 1 and 2 a preferred embodiment of a system 1 for predicting a syncope event according to the present invention comprises a sensor unit 2 adapted for continuously monitoring the pulse wave patterns 3 of a user 4, a processing unit 5 adapted for determining from the pulse wave patterns 3 a measure of the blood flow to the user's brain, said processing unit 5 being further adapted for generating an alarm signal 6, if the blood flow measure indicates the future occurrence of a syncope.

The sensor unit 2 comprises a passive (i.e. non-emitting) sensor 7 for measuring the pulse wave patterns 3 of the user 4 (step 100). The sensor 7 is located on the left carotid artery in the upper neck region 8 of the user 4. Thus the potentially excessive delays of peripheral measures at finger or wrist are eliminated with respect to a rapid drop of the supply of blood to the user's brain and the ambulatory comfort of the user 4 is preserved. Alternatively the sensor unit 2 including the sensor 7 might be positioned on the right subclavian artery (as indicated with dotted lines) or on the left subclavian artery in the shoulder region 10. As passive sensor 7 a piezo-foil transducer is used, the transducer being adapted for measuring the surface vibrations of the user's carotid arterial pulse. As an example the “Androsonix” sensor of Andromed Inc., which is designed for surface-wave recordings, can be employed.

The sensor unit 2 further comprises a wireless sender 9 and a power supply 11, all three components being encapsulated into a very light housing. The overall weight of the sensor unit 2 is about 8 g. The dimensions of the housing are about 1 cm² area and a few millimeters height. The housing is adapted to be positioned on the user's skin covering one of the carotid arteries in the upper neck region 8, e.g. using a common adhesive film or tissue (not shown). However, other methods of positioning the sensor unit 2 on the user's skin may be employed as well. For power supply 11 a battery is provided within the housing.

The measured data is transferred from the sensor unit 2 to the processing unit 5 by means of a wireless communication line 12. For this purpose the sender 9 is adapted in a way, that the wireless connection can be realized using the Bluetooth standard or other well-known techniques, for example using WIFI standards.

The processing unit 5 is adapted for performing all tasks of calculating and computing the measured data as well as determining and assessing results. This is achieved according to the invention by means of a computer software 13 comprising computer instructions adapted for carrying out the steps of the inventive method, when the software 13 is executed in the processing unit 5. The processing unit 5 itself may comprise functional modules or units, which are implemented in form of hardware, software or in form of a combination of both.

The processing unit 5 is placed within a compact light-weight housing of a pocket device 14, which will be worn by the patient 4, for example, in one of his pockets or as a belt or the like.

For receiving measured data from the sensor unit 2 the processing unit 5 is connected to a communication unit 15. Said communication unit 15 is also placed within the housing of the pocket device 14. The communication unit 15 comprises a receiver 16 adapted for receiving measured data from the sensor device 2. The transmitted data may contain the raw pulse wave pattern data or pre-processed data. In the latter case the sensor unit 2 comprises a pre-processor (not shown).

The processing unit 5 is adapted for analysing the incoming data and to track the blood flow variations (step 101). For this purpose the processing unit 5 comprises a DSP (digital signal processing) module 17. The DSP module 17 is driven by embedded software 13, which causes the DSP module 17 to compute and monitor the blood flow variations and to decide whether an alarm signal should be produced. For post-syncope analyse the processed data are recorded over a long period ( e.g. several hours) within the pocket device 14. For this purpose the processing unit 5 is connectable to a storage devices, e.g. RAM or HDD (not shown). Said storage device can also be positioned within the pocket device 14.

Before using the system 1, a tuning of the system 1 might be performed by adjusting parameter values used in the DSP software 13 such that the blood flow monitoring can be adapted to the physiological state of the user 4 and/or to his risk profile as well.

If pulse wave patterns 3 of the user 4 are continuously captured by the sensor unit 2 (step 100) and measured data are transmitted to the processing unit 5, the processing unit 5 determines a measure of the blood flow by processing said data (step 101). As a result a single characteristic value is computed as a measure of the blood flow in the upper vascular system to the user's brain.

The magnitude of the blood flow in the aortic arch depends directly on the cardiac output that is conditioned upon two main factors. The cardiac output is given by the product of the heart rate and the stroke volume defined as the amount of blood ejected by each left ventricular contraction. Hence, the basic blood flow equation is given by

Q=HR×SV

where Q denotes the cardiac output (volume/time), HR denotes the heart rate (beats/time) and SV denotes the stroke volume (volume/beat) of the user 4. The stroke volume SV depends on the contractility of the cardiac muscle and on the filling of the left ventricle insured by the return of venous blood. The major factors intervening in the occurrence of a syncope event are thus taken into account in this single equation.

The present invention relies on the above equation to perform an approximate estimation of the cerebral blood flow, using the above described sensor 7 placed on one of the carotid or subclavian arteries. Piezo-foil transducers are highly sensitive to small displacements and provide accurate pulse-wave signals when located over close-to-the-skin arteries. FIG. 4 gives an example of the signal (pulse wave pattern) recorded with such a sensor 7 placed on the right carotid of a healthy male user 4 and exhibits clear periodic pulses 19. The signal shown is measured during a time period of 3.5 seconds (illustrated by means of the legend from 0 to 7000).

Heart rate (HR) values are estimated by means of the processing unit 5 from the time-delays 18 between successive pulses 19 and have been shown to closely follow the standard ECG derived beat-to-beat RR intervals. This provides the first chronotropic term of the above equation. An estimation of the second inotropic term (SV) is achieved by means of the processing unit 5 from the shape of the pulses 19, based on the amplitude and width of the pressure waves. By combining these two estimations, a characteristic value (blood supply indicator) 21 is obtained, reflecting the relative variations over time of the blood flow to the brain of the user 4, consecutive to either heart rate variations or changes in the ejection volume. During processing an average characteristic value is generated, based on an average signal over several heart beats, e.g. based on 3 to 5 heart beats.

The processing unit 5 is further adapted for correcting motion artifacts, e.g. caused by ambulatory conditions like walking steps. For this purpose the processing unit 5 is connected to an accelerometer sensor or movement sensor (not shown) that provides information about the type of motions and the amplitude of the perturbations induced by the user's body. This allows the data processing algorithm executed in the DSP module 17 to compensate for such artifacts, by correctly identifying and eliminating these spurious variations that are not related to the arterial pulses. It is indeed unlikely that these motion artifacts would be synchronized with the heart rate but for some rare moments and, knowing when they occur in time through the motion sensor, greatly facilitates the suppression of these unwanted signal components. The accelerometer sensor or movement sensor is placed within the pocket device 14 as well. For example a piezo accelerometer sensor or another type of accelerometer sensor (e.g. of capacitive or resistive type) can be used.

The processing device 5 is further adapted for deciding whether an alarm signal 6 should be triggered (step 102). An alarm signal 6 is generated (step 103), for example, if the (average) characteristic value 21 reaches a low threshold value (critical level). The sensitivity of step 102 is tuneable to the specific risk profile of the user 4 by performing an according parameter setting for the DSP module 17. If a critical value is reached, the alarm signal 6 is generated immediately (step 104), since the loss of consciousness occurs within about 10 seconds of the cessation of cerebral blood perfusion.

In case of a critical situation the alarm signal 6 generated by the processing unit 5 causes a sound generator 22 to locally generate an alarm sound 23 to be emitted to the user 4 (step 104). The sound generator 22 is located within the housing of the pocket device 14. Alternatively the sound generator may be located outside the housing of the pocket device 14, e.g. the sound generator may be integrated into earphones or the like, which might be of advantage for elderly people with hearing problems.

In addition to the alarm sound 23 or alternatively to that sound 23 the alarm signal 6 causes the communication unit 15 of the pocket device 14 to transmit an alarm message 24, e.g. a SMS or another textual message, to a remote receiving unit 25, e.g. to a medical emergency service (step 104). For this purpose the communication unit 15 comprises a sender 26. Such a sender 26 can for example be adapted for establishing a communication line 27 via a cell phone network.

The pocket device 14 further comprises a power supply 11′. Said power supply 11′ is adapted for supplying all components contained in housing of the pocket device 14.

In a further embodiment of the invention (not shown) a one-lead ECG is recorded simultaneously in addition to the carotid or subclavian pulse signal. If the ECG signal is transmitted to the processing unit 5, this allows the processing unit 5 to compute the pulse transit time (PTT) from left ventricle to carotid or subclavian artery, based on the ECG R-Peak. PTT values are known to be correlated with the arterial blood pressure (BP) thus providing a measure of another important parameter of the hemodynamic circulation. These BP estimations could in turn be incorporated in the monitoring of the blood supply to obtain more reliable decisions. As an alternative to ECG, PCG (Phonocardiography) can be considered by using another (identical) piezo-foil transducer positioned on the heart region 28 of the user 4. By means of the processing unit 5 an equivalent PTT can then be computed between the occurrence of the first heart-sound (S1) and the carotid or subclavian pulse, from which blood pressure variations can be inferred.

The adding of an ECG channel or an audio PCG recording of the heart sounds is especially useful in case of elderly users, which are strongly affected by arterio-sclerosis. In this case, it may be that the carotid site will appear inadequate because of the weakness of the pulse signal and therefore some other arterial location can be considered, for example at the elbow, using the same sensor 7 as described above. Alternatively, the sensor unit 2 including the sensor 7 can be placed on the left or right subclavian artery in the shoulder region 10 of the user 4. This position offers the advantage that the sensor 7 would be located along and upon the clavicle bone, on the right or left side, with an enhanced mechanical stability. Concerning the pulse wave estimation, the blood flow in the subclavian artery comes from the common carotid artery, at just a few centimeter distance, hence, it will closely reflect the evolution of the blood flow to the user's brain.

It will be evident to those skilled in the art that the invention is not limited to the details of the foregoing illustrative embodiments, and that the present invention may be embodied in other specific forms without departing from the spirit or essential attributes thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein. It will furthermore be evident that the word “comprising” does not exclude other elements or steps, that the words “a” or “an” do not exclude a plurality, and that a single element, such as a computer system or another unit may fulfil the functions of several means recited in the claims. Any reference signs in the claims shall not be construed as limiting the claim concerned.

### REFERENCE NUMERALS

1 System

2 Sensor unit

3 pulse wave pattern

4 user

5 processing unit

6 alarm signal

7 sensor

8 upper neck region

9 sender

10 shoulder region

11 power supply

12 communication line

13 software

14 pocket device

15 communication unit

16 receiver

17 DSP module

18 time delay

19 pulse

20 (free)

21 characteristic value

22 sound generator

23 alarm sound

24 alarm message

25 remote unit

26 sender

27 communication line

28 heart region

1. A method of detecting and predicting a syncope event, the method comprising the steps of:
continuously obtaining (100) the pulse wave patterns (3) of a user (4),
determining (101) from the pulse wave patterns (3) a measure (21) of the blood flow to the user's brain, and
generating (103) an alarm signal (6), if the blood flow measure (21) indicates the future occurrence of a syncope.

2. The method as claimed in claim 1, characterized in that in order to obtain the pulse wave patterns (3) the pulse signal of one of the carotid or subclavian arteries is continuously monitored.

3. The method as claimed in claim 1, characterized in that the determining of the measure (21) of the blood flow is carried out based both on the heart rate and the stroke volume of the user 4.

4. The method as claimed in claim 1, characterized in that the alarm signal (6) is generated, if the blood flow falls below a given critical level.

5. The method as claimed in claim 1, characterized in that in case the alarm signal (6) is generated, an alarm sound (23) is emitted to the user (4) and/or an alarm message (24) is transmitted to a remote receiving unit (25).

6. A system (1) for detecting and predicting a syncope event, the system (1) comprising:
a sensor unit (2) adapted for continuously obtaining the pulse wave patterns (3) from the upper vascular system of a user (4),
a processing unit (5) adapted for determining from the pulse wave patterns (3) a measure (21) of the blood flow to the user's brain, and further adapted for generating an alarm signal (6), if the blood flow measure (21) indicates the future occurrence of a syncope.

7. The system (1) as claimed in claim 6, characterized in that the sensor unit (2) comprises a passive sensor (7), in particular a piezo-foil transducer adapted for measuring surface vibrations of the user's carotid arterial pulse.

8. The system (1) as claimed in claim 6, characterized in that the sensor (7) is located on the carotid artery, preferably in the upper neck region (8) of the user (4) or on the subclavian artery in the shoulder region (10) of the user (4).

9. A computer program (13) to be executed in a computer (5, 17), said computer (5, 17) being part of a system (1) for detecting and predicting a syncope event, said system (1) comprising a sensor unit (2) adapted for continuously obtaining the pulse wave patterns (3) from the upper vascular system of a user (4), said program (13) comprising computer instructions to determining from the pulse wave patterns (3) a measure (21) of the blood flow to the user's brain, when the computer program is executed in the computer (5, 17).

10. The computer program (13) as claimed in claim 9, said program (13) comprising computer instructions to generate an alarm signal (6), if the blood flow measure (21) indicates the future occurrence of a syncope, when the computer program (13) is executed in the computer (5, 17).
